(19) United States
(12) Patent Application Publication
(10) Pub. No.: US 2024/0318140 A1
(43) Pub. Date: Sep. 26, 2024

(54) SCAFFOLD FOR PRODUCING CULTURED MEAT CONTAINING BIO-CELLULOSE AND MANUFACTURING METHOD THEREOF

(71) Applicant: CELLMEAT CORP., Yangcheon-gu (KR)

(72) Inventors: Giljun PARK, Gwangju (KR); Heejung KIM, Gwangju (KR); Minsoo LEE, Gwangju (KR); Junhyuk BAE, Goyang-si (KR); Jongyoung LIM, Gwangju (KR); Yubin LEE, Gwangju (KR)

(73) Assignee: CELLMEAT CORP., Yangcheon-gu (KR)

(21) Appl. No.: 18/737,151

(22) Filed: Jun. 7, 2024

Related U.S. Application Data
(63) Continuation of application No. PCT/KR2022/019274, filed on Nov. 30, 2022.

(30) Foreign Application Priority Data
Dec. 7, 2021 (KR) 10-2021-0174094
Mar. 7, 2022 (KR) 10-2022-0028847

(51) Int. Cl. C12N 5/077; A23L 13/00

(52) U.S. Cl. CPC C12N 5/0658; A23L 13/00; C12N 2533/70; C12N 2533/74; C12N 2533/76; C12N 2533/78; C12N 2533/90

(57) ABSTRACT

The present disclosure relates to a scaffold based on microbial-derived bio-cellulose and a manufacturing method thereof, and/or a hydrogel scaffold containing microbial-derived bio-cellulose powder and a manufacturing method thereof. The scaffolds can be usefully used as scaffolds for manufacturing cultured meat due to excellent cell adhesion capability and physical properties.

### CROSS-REFERENCE TO RELATED APPLICATION

The present application is a continuation of International Patent Application No. PCT/KR2022/019274, filed on Nov. 30, 2022, which is based upon and claims the benefit of priority to Korean Patent Application Nos. 10-2021-0174094 filed on Dec. 7, 2021 and 10-2022-0028847 filed on Mar. 7, 2022. The disclosures of the above-listed applications are hereby incorporated by reference herein in their entirety.

### BACKGROUND

### 1. Technical Field

The present disclosure relates to a scaffold for producing cultured meat containing bio-cellulose or a scaffold for producing cultured meat containing bio-cellulose powder, and methods for producing the same.

### 2. Description of Related Art

It has been well known that bio-cellulose is produced through microbial culture, and is typically produced by microorganisms selected from Rhizobium sp., Agrobacter sp., Gluconacetobacter sp., and Acetobacter sp., through cell culture in a culture medium using fruits. The produced bio-cellulose has been used as pharmaceuticals like burn treatment agents and as cosmetics such as mask packs, and recently application fields of bio-cellulose are getting wider, for example, bio-cellulose is used in packing containers. The most efficient bio-cellulose producing strain is known to be from strain of Acetobacter sp. When strains of Acetobacter sp. are cultured using a stationary culture method, bio-cellulose is produced on the culture medium in the form of a translucent film. Bio-cellulose produced by the strains of Acetobacter sp. may contain 90% or more of moisture in an oxygen-mediated β1,4 glycosidic linkage and form a three-dimensional reticular structure. Additionally, it is known that the bio-cellulose produced by the strains of Acetobacter sp. is decomposed in the human body and discharged to the outside, and is harmless to the human body, so research into new fields is actively proceeding.

The extracellular matrix (ECM) is located between cells to fill spaces among a plurality of cells. The extracellular matrix primarily composed of polysaccharide gels and protein fibers fills the spaces among cells and provides a physical skeleton to structurally support animal tissues, maintain the shape of the animal tissues, and protect the animal tissues from external impacts. Similarly, since the muscle tissues are filled with the extracellular matrix between muscle cells, the extracellular matrix between muscle cells plays a vital role in maintaining the shape and structure of muscle tissues and in having physical properties.

Scientists have long conducted various studies using two-dimensional cell culture methods with petri dishes. However, when cells are cultured using the two-dimensional cell culture methods, there is a problem in that an environment different from actual tissue is created. So, methods for culturing cells using three-dimensional cell culture methods are being studied, and among them, the importance of cell-adhesion scaffolds with structures of the extracellular matrix, which are similar to actual tissues and allow for cell adhesion has been recognized. Scaffolds for creating tissues similar to the in vivo environment are used in various fields through tissue engineering technology and have recently been utilized in the field of cultured meat production.

A three-dimensional cell culture scaffold suitable for cultured meat production must satisfy conditions of biocompatibility, biostability, biofunctionality, and harmlessness to human body, and the physical properties and structure of the scaffolds should be similar to the structure of actual meat. Additionally, considering animal protection and welfare, the need for cultured meat scaffolds made from non-animal sources is emerging. Meanwhile, cellulose derived from plants and algae has been extensively studied and used in various fields. Research on scaffolds that allow for cell adhesion or culture using the cellulose derived from plants and algae is ongoing. However, it is difficult to adjust the internal structure of the scaffold since the cellulose derived from plants and algae uses tissues, there is a limitation in using the cellulose derived from plants and algae as a scaffold for cultured meat production due to the complexity of the decellularization process.

Accordingly, the inventors of the present disclosure have made efforts to manufacture a scaffold for cultured meat production based on microbial-derived bio-cellulose, rather than cellulose derived from plants or algae. As a result, it has been confirmed that the scaffold based on microbial-derived bio-cellulose is harmless to the human body, is edible, and is suitable for replicating actual tissues due to excellent cell adhesion properties. Furthermore, by powdering and adding microbial-derived bio-cellulose, the inventors of the present disclosure have completed the present disclosure by manufacturing a scaffold for producing cultured meat, which can control the properties of bio-cellulose and solve the problem that it took lots of time to produce large volumes of bio-cellulose.

### SUMMARY

An objective of the present disclosure is to provide a scaffold for producing cultured meat containing microbial-derived bio-cellulose.

Another objective of the present disclosure is to provide a manufacturing method of the scaffold for producing cultured meat.

Another objective of the present disclosure is to provide cultured meat produced by culturing non-human animal-derived cells on the scaffold manufactured by the manufacturing method.

Another objective of the present disclosure is to provide a composition for manufacturing a cultured meat scaffold, which includes microbial-derived bio-cellulose powder, a natural polymer gelling agent, and a gelation accelerator.

Another objective of the present disclosure is to provide a cultured meat scaffold manufactured from the composition.

Another objective of the present disclosure is to provide a manufacturing method of a cultured meat scaffold, including: (a) obtaining bio-cellulose by static culturing of microorganisms in a medium containing peptone; (b) washing the bio-cellulose obtained in operation (a) with distilled water and drying the washed bio-cellulose; (c) freezing the dried bio-cellulose, and crushing the bio-cellulose at 10,000 to 15,000 rpm to obtain microbial-derived bio-cellulose powder; (d) mixing the microbial-derived bio-cellulose powder with a natural polymer gelling agent; and (e) freeze-drying the product of operation (d) and adding a gelation accelerator.

Another objective of the present disclosure is to provide cultured meat produced by culturing non-human animal-derived cells on the scaffold manufactured by the method.

Advantages and features of the present disclosure and methods accomplishing the advantages and features will become apparent from the following detailed description of exemplary embodiments with reference to the accompanying drawings. However, the present disclosure is not limited to exemplary embodiment disclosed herein but will be implemented in various forms. The exemplary embodiments are provided so that the present disclosure is completely disclosed, and a person of ordinary skilled in the art can fully understand the scope of the present disclosure. Therefore, the present disclosure will be defined only by the scope of the appended claims.

Terms used in the specification are used to describe specific embodiments of the present disclosure and are not intended to limit the scope of the present disclosure. In the specification, the terms of a singular form may include plural forms unless otherwise specified. It should be also understood that the terms of ‘include’ or ‘have’ in the specification are used to mean that there is no intent to exclude existence or addition of other components besides components described in the specification. In the detailed description, the same reference numbers of the drawings refer to the same or equivalent parts of the present disclosure, and the term “and/or” is understood to include a combination of one or more of components described above. It will be understood that terms, such as “first” or “second” may be used in the specification to describe various components but are not restricted to the above terms. The terms may be used to discriminate one component from another component. Therefore, of course, the first component may be named as the second component within the scope of the present disclosure.

Unless otherwise defined, all terms (including technical and scientific terms) used herein have the same meaning as commonly understood by those skilled in the technical field to which the present disclosure pertains. It will be further understood that terms, such as those defined in commonly used dictionaries, should not be interpreted in an idealized or overly formal sense unless expressly so defined herein.

The present disclosure provides a scaffold for producing cultured meat containing microbial-derived bio-cellulose.

The cultured meat of the present disclosure is also referred to as clean meat, cell-based meat, or cultivated meat, is fit for consumption by humans or non-human animals, and is edible.

The scaffold of the present disclosure is a scaffold based on microbial-derived bio-cellulose, has a structure similar to the structure of meat, and is a scaffold for producing cultured meat, which has cell adhesion capability. More specifically, the scaffold based on microbial-derived bio-cellulose has excellent cell adhesion capability and can have the structure similar to chicken, beef, or pork.

According to an embodiment of the present disclosure, the microorganisms are one or more types selected from a group consisting of Acetobacter sp., Gluconacetobactersp., Rhizobium sp., and Agrobacter sp., and preferably, may be Acetobacter sp. or Gluconacetobacter sp., which are known for good production yields.

According to another embodiment of the present disclosure, the microbial-derived bio-cellulose can be produced in a liquid medium containing animal peptone, bacterial peptone, or plant peptone.

The animal peptone may be meat and/or casein.

The bacterial peptone may be Bacto peptone.

The plant peptone may be one or more types selected from a group consisting of soy peptone, wheat peptone, broadbean peptone, potato peptone, pea peptone, papaic soy peptone, and lupin peptone, and preferably, may be soy peptone.

The plant peptone may be a product obtained by breaking down proteins derived from plants through acid treatment, alkali treatment, enzymatic treatment, heat treatment, or high-pressure treatment.

According to another embodiment of the present disclosure, the microbial-derived bio-cellulose can be produced in a liquid medium containing glucose, fructose, or a combination thereof as carbon sources.

Strains producing typically use glucose, sucrose, molasses, fructose, mannitol, and glycerol as carbon sources during growth, and use persimmon juice, vinegar, apple juice, grape juice, brewery waste, and coconut by-products as auxiliary carbon sources, and use casein, peptone, and yeast extract as nitrogen sources. However, the optimal carbon sources, the auxiliary carbon sources, and the nitrogen sources vary depending on the strains.

Selecting the optimal types of carbon sources, auxiliary carbon and nitrogen sources, sources and establishing culture conditions with appropriate concentration are crucial not only from a cost perspective but also for yield improvement by preventing yield reduction due to nutrient deficiencies and inhibiting the accumulation of by-products triggered by excessive consumption of carbon sources.

In a specific embodiment of the present disclosure, the bio-cellulose can be produced in a liquid medium containing glucose and fructose.

In a specific embodiment of the present disclosure, the scaffold for producing cultured meat provides a structure of the bio-cellulose that gets more minute as the content of glucose increases but gets looser as the content of fructose increases. The bio-cellulose may be produced in a liquid medium containing glucose and fructose in a ratio ranging from 5:1 to 1:5.

In the present disclosure, when producing microbial-derived bio-cellulose using strains of Gluconacetobacter Xylinus subsp. Xylinus (KCCM41431), which has been confirmed to produce bacterial cellulose, the scaffold for producing cultured meat of the present disclosure has been designed through studying the optimal culture conditions for carbon sources, such as glucose and fructose alone or in a mixture ratio, to have physical properties of the scaffold for manufacturing cultured meat.

Furthermore, the present disclosure provides a manufacturing method of a scaffold for producing cultured meat includes the operations: following (a) introducing microorganisms into a liquid medium containing peptone and statically culturing the microorganisms at 28 to 32° C. to obtain bio-cellulose; and (b) washing the bio-cellulose obtained in operation (a) with distilled water, boiling the bio-cellulose in an alkaline solution, then washing the bio-cellulose again with distilled water to manufacture a hydrogel-type bio-cellulose scaffold.

According to an embodiment of the present disclosure, in the manufacturing method, the liquid medium in operation (a) may include glucose, fructose, or a combination thereof as carbon sources.

According to another embodiment of the present disclosure, the manufacturing method may further include freeze-drying the hydrogel-type bio-cellulose scaffold of operation (b) to manufacture a freeze-dried type bio-cellulose scaffold.

According to another embodiment of the present disclosure, in the manufacturing method, the microorganisms may be one or more types selected from a group consisting of Acetobacter sp., Gluconacetobacter sp., Rhizobium sp., and Agrobacter sp.

According to another embodiment of the present disclosure, in the manufacturing method, peptone may be animal peptone, bacterial peptone, or plant peptone.

The animal peptone may be meat and/or casein.

The bacterial peptone may be Bacto peptone.

According to another embodiment of the present disclosure, in the manufacturing method, the plant peptone may be one or more types selected from a group consisting of soy peptone, wheat peptone, broadbean peptone, potato peptone, pea peptone, papaic soy peptone, and lupin peptone.

According to another embodiment of the present disclosure, in the manufacturing method, the plant peptone may be a product obtained by breaking down proteins derived from plants through acid treatment, alkali treatment, enzymatic treatment, heat treatment, or high-pressure treatment.

Furthermore, the present disclosure provides cultured meat produced by culturing non-human animal-derived cells on the scaffold manufactured by the method.

According to another embodiment of the present disclosure, the non-human animal-derived cells may be one or more types selected from a group consisting of muscle stem cells, muscle cells, and their progenitors.

The muscle stem cells may be satellite cells or myoblasts, for example, myoblasts, but are not limited thereto.

The muscle cells may be myocytes or myotubes, for example, myocytes, but are not limited thereto.

The progenitors refer to cells capable of producing cells differentiated into a plurality of systems, such as myoblasts, fibroblasts, adipocytes, stromal cells, dermal cells, smooth muscle cells, and endothelial cells. The progenitors are distinct from stem cells in that they typically do not have a broad capacity for self-replication.

The non-human animal-derived cells may further include fat cells or adipocytes and/or their progenitors, stromal cells (connective tissue) and/or their progenitors, or endothelial cells (vascular) and/or their progenitors, but are not limited thereto.

According to an embodiment of the present disclosure, the non-human animals may be selected from a group consisting of cattle, sheep, pigs, poultry, crustaceans, and fish, but are not limited thereto.

Furthermore, the present disclosure provides a composition for manufacturing a scaffold for producing cultured meat containing microbial-derived bio-cellulose powder, a natural polymer gelling agent, and a gelation accelerator.

The microorganisms of the present disclosure may be one or more types selected from a group consisting of Rhizobium sp., Agrobacter sp., Gluconacetobacter sp., and Acetobacter sp. Preferably, it may be Acetobacter sp. or Gluconacetobacter sp. Preferably, the microorganisms may be selected from Acetobacter sp. or Gluconacetobacter sp. with excellent production yields, but are not limited thereto.

The cultured meat of the present disclosure is also referred to as clean meat, cell-based meat, or cultured meat, and is referred to as clean meat, cell-based meat, or cultivated meat, is fit for consumption by humans or non-human animals, and is edible.

The scaffold of the present disclosure is a scaffold based on microbial-derived bio-cellulose powder, natural polymer gelling agent, and gelation accelerator, has a structure similar to the structure of meat, and is a scaffold for producing cultured meat, which has cell adhesion capability.

The bio-cellulose of the present disclosure can be produced in a medium containing animal peptone, bacterial peptone, or plant peptone, and, preferably, may be plant peptone, but is not limited thereto.

The animal peptone may be meat and/or casein.

The bacterial peptone may be Bacto peptone.

The plant peptone may be one or more types selected from a group consisting of soy peptone, wheat peptone, broadbean peptone, potato peptone, pea peptone, papaic soy peptone, and lupin peptone, and preferably, may be soy peptone, but is not limited thereto.

Strains producing typically use glucose, sucrose, molasses, fructose, mannitol, and glycerol as carbon sources during growth, and use persimmon juice, vinegar, apple juice, grape juice, brewery waste, and coconut by-products as auxiliary carbon sources, and use casein, peptone, and yeast extract as nitrogen sources. However, the optimal carbon sources, the auxiliary carbon sources, and the nitrogen sources vary depending on the strains.

Selecting the optimal types of carbon sources, auxiliary carbon sources, and nitrogen sources and establishing culture conditions with appropriate concentration are crucial not only from a cost perspective but also for yield improvement by preventing yield reduction due to nutrient deficiencies and inhibiting the accumulation of by-products triggered by excessive consumption of carbon sources.

In the present disclosure, when producing microbial-derived bio-cellulose using strains of Gluconacetobacter Xylinus subsp. Xylinus (KCCM41431), which has been confirmed to produce bacterial cellulose, the scaffold for producing cultured meat of the present disclosure has been designed through studying the optimal culture conditions for carbon sources, such as glucose and fructose alone or in a mixture ratio, to have physical properties of the scaffold for manufacturing cultured meat.

The natural polymer gelling agents (hydrogel raw materials) in the present disclosure may include one selected from a group consisting of alginate, agar, and carrageenan, but are not limited thereto.

Alginate, in the present disclosure, is a sticky mucous substance extracted from brown seaweeds such as sea mustard and kelp, is not soluble in water but swells in water, and is a complex polysaccharide that colloids, namely, microparticles are coagulated or dispersed in a gas or a liquid. Alginate is used for various purposes including as a thickening agent, emulsion stabilizer, and gelling agent, and can be applied in diverse fields such as food, industry, and medicine. In a natural state, alginate is combined with sodium, calcium, potassium, etc., and when included in cosmetics, has excellent adsorption properties effective for toxin removal and excellent moisturizing characteristics. When divalent metals such as Ca²⁺ are added and alginate is cross-linked to produce hydrogel, the tissues of actual organisms can be imitated.

Agar used in the present disclosure is extracted from Gracilaria seaweed, is a material obtained by freeze-drying the agar of Gracilaria, contains galactose as a main ingredient, and is a transparent material having physical properties similar to gelatin. Agar is used as an edible substance when dissolved in water and used as an industrial material. Agar dissolves well in water, and is transparent and soft. Moreover, agar is also used in the preparation of solid media for immobilizing and culturing microorganisms, plant tissues, and animal cells. As agar is a low-calorie, high dietary fiber material, it holds potential as a substitute for fats and proteins.

Carrageenan in the present disclosure is extracted from red algae, primarily consists of galactose, is widely used as a gelling and thickening agent in food, and is also widely used as a stabilizer or dispersant in pharmaceuticals and cosmetics. Carrageenan completely dissolves in water at temperatures of 70 to 80° C. and above. Carrageenan is a light ivory-colored, odorless, tasteless powder. Aqueous solutions in which carrageenan is dissolved form viscous solutions or gels at room temperature, and exhibit excellent water retention, thereby maintaining viscosity over time.

The gelling accelerator in the present disclosure can be group 1 or group 2 metal salts. The gelling accelerator may be one or more metal salts selected from a group consisting of potassium chloride, calcium chloride, magnesium chloride, and calcium lactate, but is not limited thereto.

The microbial-derived bio-cellulose powder in the present disclosure is obtained by freezing the microbial-derived bio-cellulose and then grinding the microbial-derived bio-cellulose at 10,000 to 15,000 rpm, and sieving through a mesh of 0.5 to 2 mm.

The composition for manufacturing a scaffold for cultured meat production of the present disclosure may comprise, with respect to 100 parts by weight of the composition, 1 to 20 parts by weight of the microbial-derived bio-cellulose powder, preferably 1 to 10 parts by weight, 1 to 5 parts by weight of the natural polymer gelling agent, preferably 1 to 2 parts by weight, and 1 to 5 parts by weight of the gelling accelerator, preferably 1 to 2 parts by weight, but is not limited thereto.

If the amount of microbial-derived bio-cellulose powder is less than 1 parts by weight relative to 100 parts by weight of the composition, the strength of the hydrogel is very weak, and there is a limitation in number of cells that can be cultured. If the amount of microbial-derived bio-cellulose powder exceeds 20 parts by weight, the cellulose powder interferes with the cross-linking of the hydrogel components, making it very difficult to maintain the form of the hydrogel.

If the natural polymer gelling agent is less than 1 parts by weight relative to 100 parts by weight of the composition, it difficult to maintain the form of the hydrogel due to insufficient material for cross-linking. If the natural polymer gelling agent exceeds 5 parts by weight, a very hard hydrogel is formed, and the pore size of the hydrogel decreases, reducing water absorption and complicating cell culture.

Furthermore, the present disclosure provides a cultured meat scaffold manufactured from the composition for manufacturing a cultured meat scaffold.

The cultured meat scaffold may include microbial-derived bio-cellulose powder and natural polymer gelling agents.

As the concentration of bio-cellulose powder in the composition increases, the hardness, gumminess, and chewiness of the cultured meat scaffold can increase.

Furthermore, the elasticity, cohesiveness, and resilience of the cultured meat scaffold may decrease as the concentration of bio-cellulose powder in the composition increases.

Additionally, the present disclosure provides a manufacturing method of the cultured meat scaffold.

The manufacturing method of the cultured meat scaffold can include the following operations: (a) statically culturing microorganisms in a medium containing peptone to obtain bio-cellulose; (b) washing the bio-cellulose obtained in operation (a) with distilled water and drying it; (c) freezing the dried bio-cellulose, then grinding it at 10,000 to 15, 000 rpm to obtain microbial-derived bio-cellulose powder; (d) mixing the microbial-derived bio-cellulose powder with a natural polymer gelling agent; and (e) freeze-drying the mixture from operation (d) and adding a gelling accelerator.

The peptone can be one or more types selected from a group consisting of soy peptone, wheat peptone, broadbean peptone, potato peptone, pea peptone, papainic soy peptone, and lupin peptone, but is not limited thereto.

The microorganisms can be one or more types selected from a group consisting of Rhizobium sp., Agrobacter sp., Gluconacetobacter sp., and Acetobacter sp., preferably, Acetobacter sp. or Gluconacetobacter sp., but are not limited thereto.

The natural polymer gelling agent can include any one selected from a group consisting of alginate, agar, and carrageenan, but is not limited thereto.

The gelling accelerator may be one or more metal salts selected from a group consisting of potassium chloride, calcium chloride, magnesium chloride, and calcium lactate, but is not limited thereto.

Moreover, the present disclosure provides cultured meat produced by culturing non-human animal-derived cells on the cultured meat scaffold manufactured by the described method.

The non-human animal-derived cells may be selected from a group consisting of muscle stem cells, muscle cells, and their progenitors.

The muscle stem cells can be satellite cells or myoblasts, for example, myoblasts, but are not limited thereto.

The muscle cells can be myocytes or myotubes, for example, myocytes, but are not limited thereto.

The progenitors refer to cells capable of producing cells differentiated into a plurality of systems, such as myoblasts, fibroblasts, adipocytes, stromal cells, dermal cells, smooth muscle cells, and endothelial cells. The progenitors are distinct from stem cells in that they typically do not have a broad capacity for self-replication.

The non-human animal-derived cells may further include fat cells or adipocytes and/or their progenitors, stromal cells (connective tissue) and/or their progenitors, or endothelial cells (vascular) and/or their progenitors, but are not limited thereto.

According to an embodiment of the present disclosure, the non-human animals may be selected from a group consisting of cattle, sheep, pigs, poultry, crustaceans, and fish, but are not limited thereto.

### BRIEF DESCRIPTION OF THE DRAWINGS

FIG. 1 is a view illustrating a bio-cellulose scaffold of a hydrogel form manufactured in a liquid medium containing 2 parts by weight of glucose relative to 100 parts by weight of Acetobacter liquid medium or a bio-cellulose scaffold of a freeze-dried form manufactured by freeze-drying hydrogel.

FIG. 2 illustrates an image obtained by freeze-drying the bio-cellulose scaffold of the hydrogel form manufactured in the liquid medium containing 2 parts by weight of glucose or fructose relative to 100 parts by weight of Acetobacter liquid medium and observing a cross-section of the hydrogel bio-cellulose scaffold with a scanning electron microscope (SEM).

FIG. 3 illustrates an image obtained by observing adhesion of muscle stem cells on the bio-cellulose scaffold of the hydrogel form manufactured in the liquid medium containing 2 parts by weight of glucose or fructose relative to 100 parts by weight of Acetobacter liquid medium with the SEM.

FIG. 4 illustrates an image obtained by freeze-drying the bio-cellulose scaffold of the hydrogel form manufactured in the liquid medium containing 2 parts by weight of carbon sources, which contain glucose and fructose mixed at mixture ratios of 5:1, 3:1, 1:1, 1:3, and 1:5, relative to 100 parts by weight of Acetobacter liquid medium, and observing a cross-section of the hydrogel bio-cellulose scaffold with the SEM.

FIG. 5 illustrates an image obtained by observing adhesion of muscle stem cells on the bio-cellulose scaffold of the hydrogel form manufactured in the liquid medium containing 2 parts by weight of carbon sources, which contain glucose and fructose mixed at mixture ratios of 5:1, 3:1, 1:1, 1:3, and 1:5, relative to 100 parts by weight of Acetobacter liquid medium with the SEM.

FIG. 6 is a mimetic diagram illustrating a method for tertiary grinding of bio-cellulose.

FIG. 7 illustrates a scaffold for cultured meat manufactured by mixing powdered bio-cellulose, alginate, agar, or carrageenan.

FIG. 8A is a view for verifying the adhesion and culture of muscle stem cells on a scaffold made from alginate and a scaffold that bio-cellulose powder is added to alginate, FIG. 8B is a view for verifying the adhesion and culture of muscle stem cells on a scaffold made from agar and a scaffold that bio-cellulose powder is added to agar, and FIG. 8C is a view for verifying the adhesion and culture of muscle stem cells on a scaffold made from carrageenan and a scaffold that bio-cellulose powder is added to carrageenan.

FIG. 9 is a view illustrating the results of tracking cell culture capacity through a ratio of fluorescent staining to total area.

FIG. 10 is a view analyzing the cross-sections of scaffolds containing alginate, agar or carrageenan and the cross-sections of scaffolds in which alginate, agar, or carrageenan is mixed with bio-cellulose powder.

FIG. 11 is a view illustrating a scaffold manufactured by mixing 0, 1, 5, 10 parts by weight of bio-cellulose powder and 2 parts by weight of alginate, agar, or carrageenan, relative to 100 parts by weight of the total composition.

FIG. 12 illustrates the results of hardness, gumminess, chewiness, springiness, cohesiveness, and resilience for chicken, beef, and pork, and changes in hardness, gumminess, chewiness, springiness, cohesiveness, and resilience depending on concentrations of bio-cellulose powder in the bio-cellulose mixed hydrogel scaffold.

### DETAILED DESCRIPTION

Hereinafter, the present disclosure will be described in more detail with reference to the following embodiments and experimental examples. However, the scope of the present disclosure is not limited to the embodiments and experimental examples, and includes modifications that are equivalent to the technical idea described herein.

### Embodiment 1. Manufacture of Microbial-Derived Bio-Cellulose Scaffold

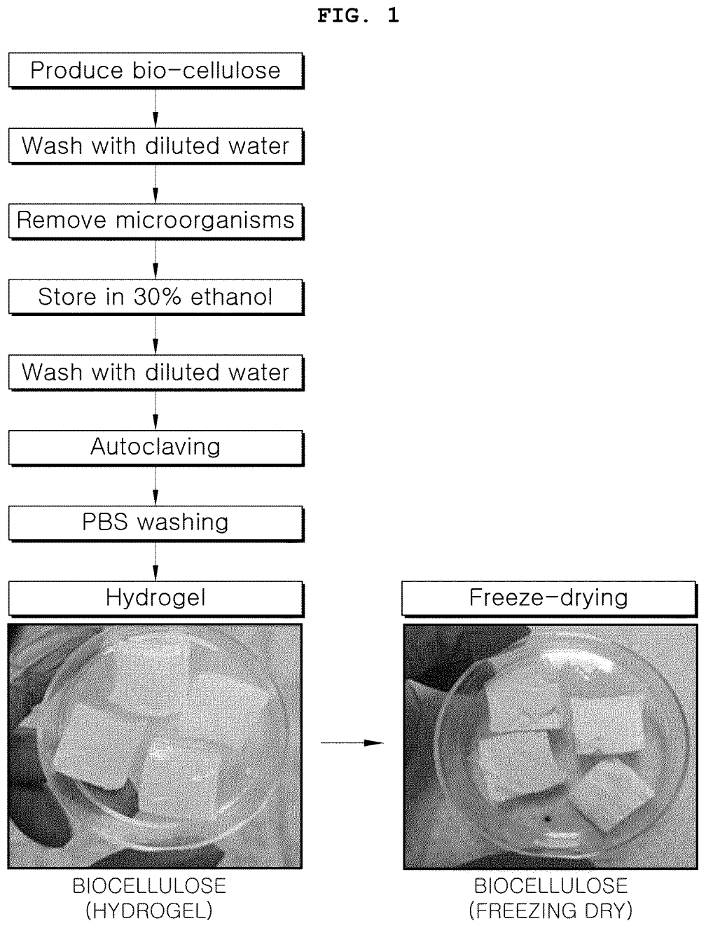

To manufacture a bio-cellulose scaffold, a Gluconacetobacter strain, specifically Gluconacetobacter Xylinus subsp. Xylinus (strain number KCCM41431), was obtained in live form from the Korean Culture Center of Microorganisms. The strain, stored at −80° C., was smeared in a Gluconacetobacter solid medium (containing 0.5% yeast extract, 0.5% soy peptone, 2% glucose, 0.27% disodium phosphate, 0.15% citric acid, and 2% agar) and cultured at 30° C. for 48 hours. The Gluconacetobacter strain cultured in the solid medium was inoculated into Gluconacetobacter liquid medium (containing 0.5% yeast extract, 0.5% soy peptone, 2% glucose, 0.27% disodium phosphate, and 0.15% citric acid), and then, statically cultured for over 72 hours to obtain bio-cellulose. To hydrogelate the obtained bio-cellulose, the medium embedded within the bio-cellulose was washed with distilled water. To remove remaining microorganisms, the bio-cellulose was boiled in a 0.1 M NaOH solution for one hour. After boiling, the bio-cellulose was washed with distilled water three times to remove the NaOH solution, and the hydrogel form of the bio-cellulose scaffold was stored in 30% ethanol. The stored bio-cellulose scaffold was washed with distilled water, and then, sterilized through autoclaving. The sterilized bio-cellulose scaffold was washed with PBS three times to produce both a hydrogel form bio-cellulose scaffold and a freeze-dried form bio-cellulose scaffold (FIG. 1). Under the same conditions, a bio-cellulose scaffold was also manufactured using meat peptone instead of soy peptone.

### Embodiment 2. Comparison of Bio-Cellulose Scaffold According to Types of Carbon Sources

### 2-1. Cross-Sectional Analysis of Bio-Cellulose Scaffold According to Types of Carbon Sources

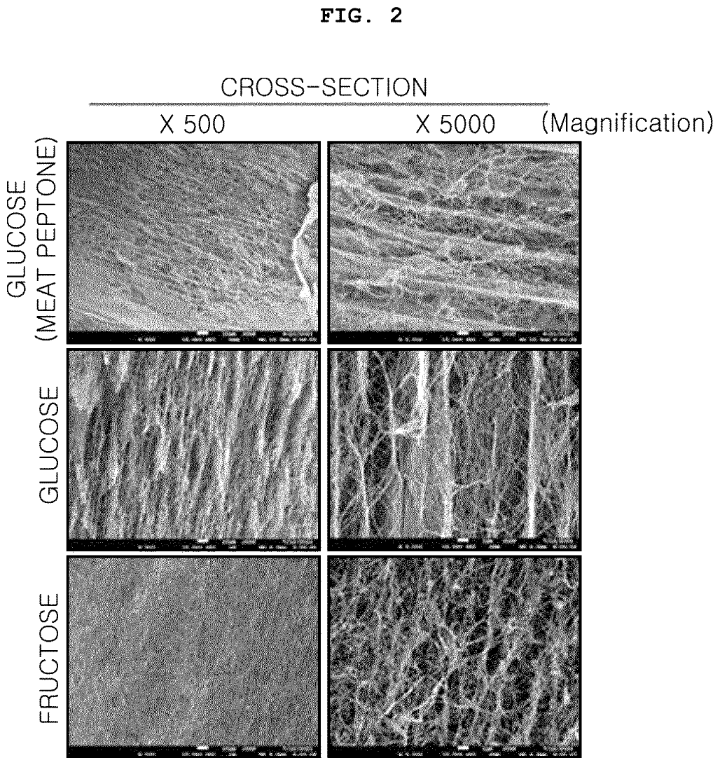

To observe the cross-section of the hydrogel form bio-cellulose scaffold manufactured in the liquid medium containing 2 parts by weight of the carbon sources, which contain glucose or fructose, relative to 100 parts by weight of Acetobacter liquid medium, the hydrogel form bio-cellulose scaffold was pre-frozen at −80° C. for 24 hours, and then, freeze-dried for 48 hours. The freeze-dried bio-cellulose scaffold was platinum-coated once at 20 mA for 60 seconds, and then, the cross-section of the coated bio-cellulose scaffold was observed through SEM (FIG. 2).

As a result, it was confirmed that the cross-section of the bio-cellulose scaffold manufactured using meat peptone and glucose as the carbon sources was similar to the texture of meat having hard layers, and formed a very tough and rigid structure with multiple fine layers. It was confirmed that bio-cellulose produced using soy peptone and glucose had the same structure as the bio-cellulose scaffold manufactured using meat peptone and glucose. In contrast, the cross-section of the bio-cellulose scaffold manufactured using fructose as the carbon sources did not form hard layers due to the sponge-like structure which was not minute, and was not relatively rigid and tough compared with the bio-cellulose scaffold manufactured using glucose as the carbon sources.

Since both of the bio-cellulose scaffolds had structures similar to the texture of meat, it was expected that cultured meat similar to the texture of meat could be produced if muscle cells were adhered on the bio-cellulose scaffold of the present disclosure.

### 2-2. Cell Adhesion Analysis of Bio-Cellulose Scaffolds According to Types of Carbon Sources

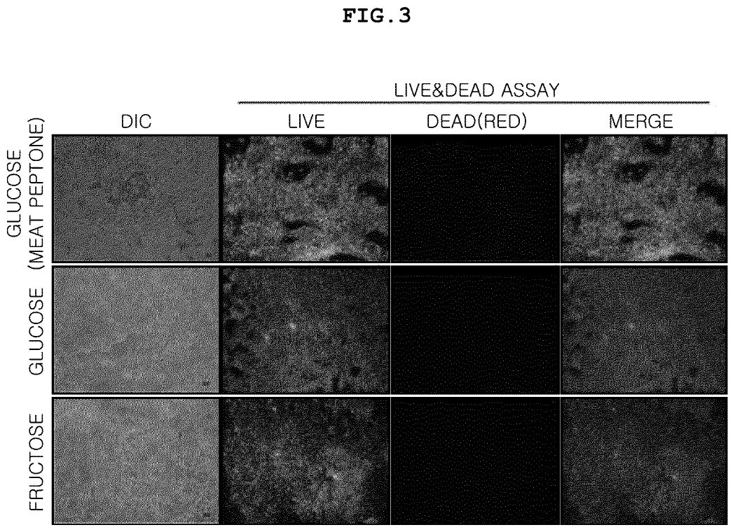

To evaluate the suitability of microbial-derived bio-cellulose produced with glucose or fructose as carbon sources to be used as muscle cell culture scaffolds, muscle stem cells were seeded onto the hydrogel form bio-cellulose scaffold. These muscle stem cells, derived from non-human animal sources such as chicken, pig, or bovine muscle tissues, were directly isolated through primary cell culture. The muscle stem cells were cultured for three hours, and then, a culture medium was added to adhere the muscle stem cells onto the scaffold well. After further culture for 24 hours, the viability and adhesion of the muscle stem cells on the bio-cellulose scaffold were observed through a fluorescence microscope, using the LIVE & DEAD assay (Thermofisher L3223), which stains living cells green and dead cells red, observed under a fluorescence microscope (FIG. 3).

As results, it was confirmed through the cells stained into green that the muscle stem cells were adhered and cultured on the bio-cellulose scaffold manufactured using glucose or fructose as carbon sources and the bio-cellulose scaffold manufactured using meat peptone, and it was also confirmed that over 90% of the cells were successfully adhered. Additionally, through the above results, it was demonstrated that replacing meat peptone with soy peptone does not alter the cell adhesion capability of the produced bio-cellulose.

2-3. Cross-Sectional Analysis of Bio-Cellulose Scaffolds According to Carbon Source Mixture ratios

Assuming that there is a difference between the structure of the bio-cellulose scaffolds manufactured using glucose or fructose as carbon sources and actual meat, the bio-cellulose scaffold having physical properties similar to actual meat was designed by adjusting the mixture ratio of glucose for producing bio-cellulose having a fine structure and fructose for producing bio-cellulose having a looser structure.

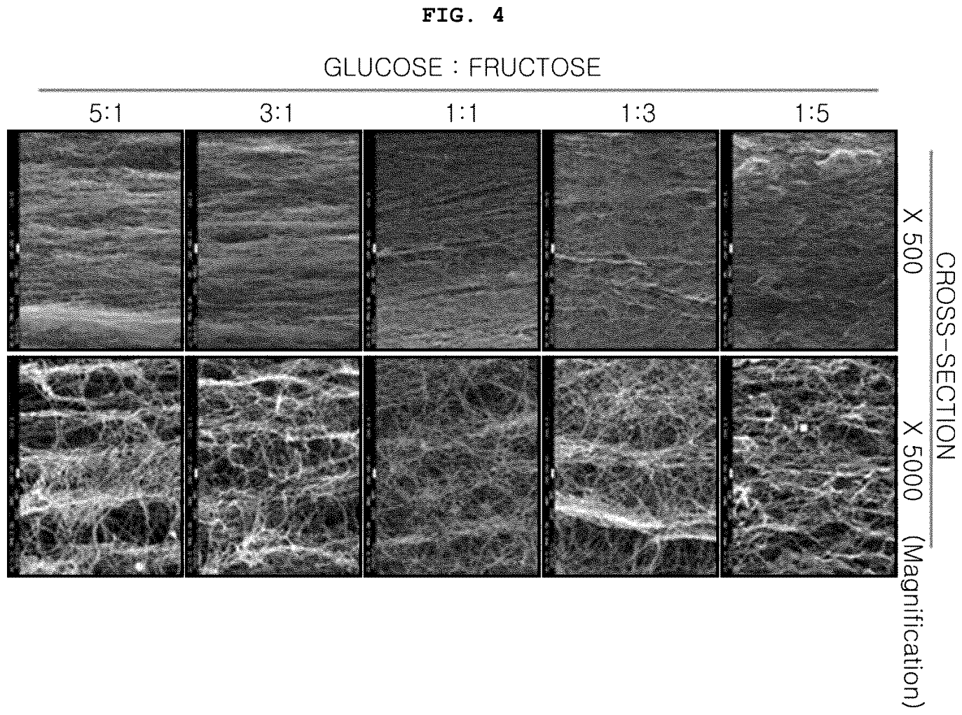

A strain of Acetobacter sp. was inoculated to an Acetobacter liquid medium manufactured by adjusting mixture ratios (Glucose:fructose=5:1, 3:1, 1:1, 1:3, and 1:5) of glucose and fructose as carbon sources, which had 2 parts by weight relative to 100 parts by weight of Acetobacter liquid medium, and then, statically cultured at 30° C. for over 72 hours to produce hydrogel form bio-cellulose scaffolds. The cross-section of each scaffold was observed through scanning electron microscopy (SEM) (FIG. 4).

Through the cross-sectional analysis of the bio-cellulose scaffolds manufactured at different mixture ratios of glucose and fructose as carbon sources, it was confirmed that as the content of glucose increased, the bio-cellulose layers were more distinctly formed with multiple layers. Furthermore, it was also confirmed that all of the bio-cellulose scaffolds manufactured by adjusting mixture ratios (Glucose:fructose=5:1, 3:1, 1:1, 1:3, and 1:5) of glucose and fructose had the structure similar to the muscular texture of meat, but the degree of fineness of the internal structures varied.

### 2-4. Analysis of Cell Adhesion of Bio-Cellulose Scaffolds According to Mixture Ratios of Carbon Source

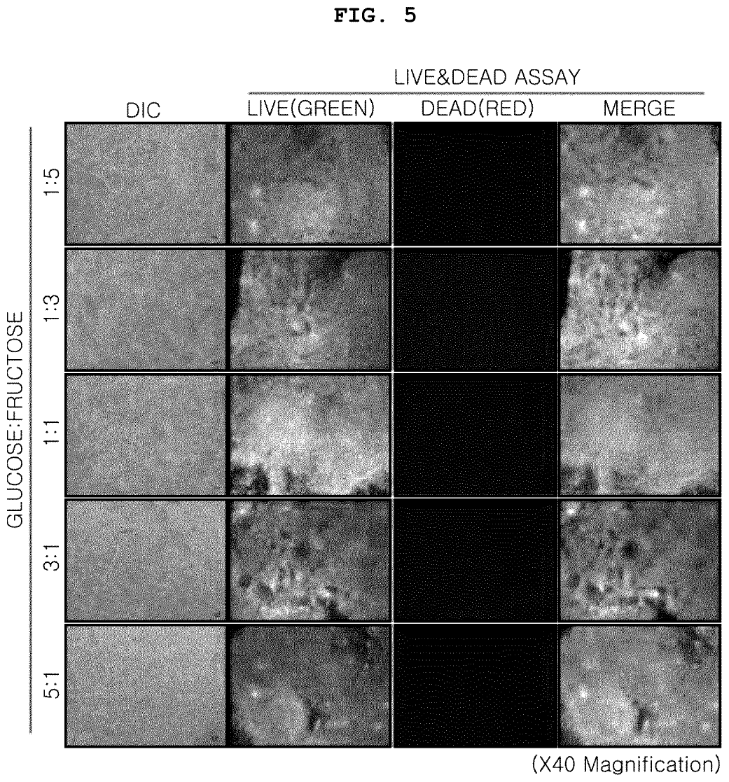

In the same way as the embodiment 2-3, muscle stem cells were seeded onto the bio-cellulose scaffolds manufactured by adjusting mixture ratios (Glucose:fructose=5:1, 3:1, 1:1, 1:3, and 1:5) of glucose and fructose as carbon sources, and cultured for three hours, and then, additional culture medium was added to ensure proper adhesion of the cells to the scaffolds. After an additional 24 hours of culture, the adhesion capability of the muscle stem cells on the bio-cellulose scaffolds was analyzed using a fluorescence microscope with the LIVE & DEAD ASSAY (Thermofisher L3223) (FIG. 5).

As a result, it was confirmed that both of the bio-cellulose scaffolds manufactured by adjusting mixture ratios (Glucose:fructose=5:1, 3:1, 1:1, 1:3, and 1:5) of glucose and fructose as carbon sources had cell adhesion capability.

### Embodiment 3. Production and Grinding of Bio-Cellulose

To produce bio-cellulose, the Gluconacetobacter xylinus subsp. Xylinus strain (deposit number: KCCM41431) was obtained in a live form from the Korean Culture Center of Microorganisms.

The strain, stored at −80° C., was smeared in Gluconacetobacter solid medium (containing 0.5% yeast extract, 0.5% soy peptone, 2% glucose, 0.27% disodium phosphate, 0.15% citric acid, and 2% agar) and cultured for 48 hours.

The cultured strain was then inoculated into Gluconacetobacter liquid medium (containing 0.5% yeast extract, 0.5% soy peptone, 2% glucose, 0.27% disodium phosphate, and 0.15% citric acid) and statically cultured for over 72 hours to obtain bio-cellulose. The obtained bio-cellulose was washed with running distilled water three times to remove the culture medium. To eliminate microorganisms, the bio-cellulose was boiled in 0.1M NaOH solution for one hour. To remove 0.1M NaOH solution, the bio-cellulose was washed with distilled water three times, and then stored in 30% ethanol.

To dry the stored bio-cellulose, the stored bio-cellulose was washed with distilled water three times to remove the ethanol and then dried in a Dryoven at 50° C. for 24 hours. The dried bio-cellulose made grinding difficult since getting very tough and hard once moisture was removed.

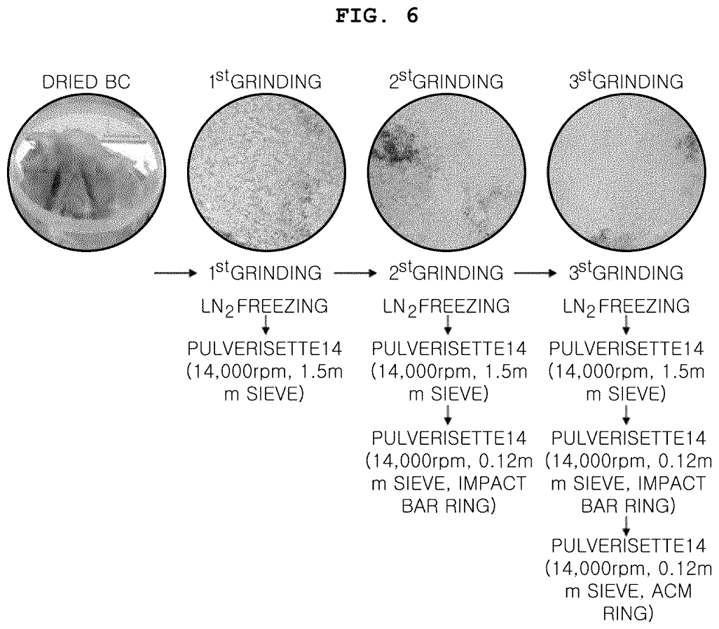

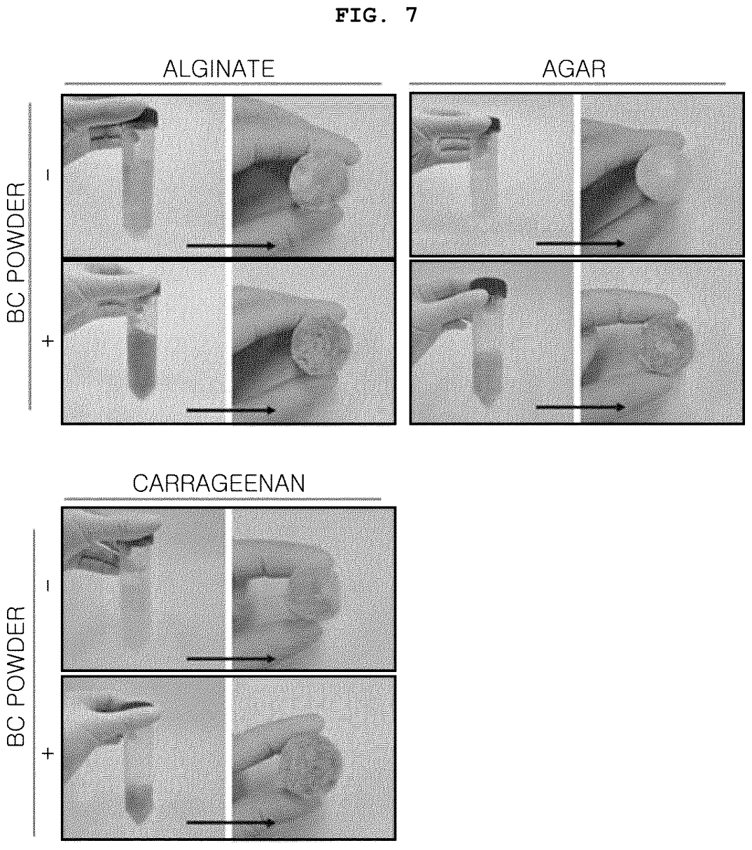

To overcome the properties of the dried bio-cellulose and grind the dried bio-cellulose, the bio-cellulose was treated with liquid nitrogen, and then subjected to a primary grinding at 14,000 rpm using a rotor mill (Pulverisette 14) with a 1.5 mm sieve. To further reduce the particle size, a secondary grinding was performed. The bio-cellulose from the primary grinding was again frozen with liquid nitrogen. To increase the grinding level of the rotor mill, the sieve was replaced with an impact bar ring, the bio-cellulose was ground at 14,000 rpm, and then, sieved through a 0.12 mm sieve to complete the secondary grinding. For additional grinding, the bio-cellulose powder from the secondary grinding was again frozen with liquid nitrogen, ground at 14,000 rpm after replacing the impact bar ring with an ACM ring, and then, sieved through a 0.12 mm sieve to finalize the third grinding (FIG. 6).

### Embodiment 4. Manufacture of Bio-Cellulose-Hydrogel Composite Scaffold

A new scaffold for cultured meat production was manufactured by mixing the bio-cellulose powdered by the method of the embodiment 3, and conventional hydrogel materials such as alginate, agar, or carrageenan (FIG. 7).

To manufacture the alginate-bio-cellulose composite scaffold, 2 parts by weight of alginate relative to 100 parts by weight of the total composition was dissolved in distilled water at room temperature for 30 minutes, and various concentrations of bio-cellulose powder were mixed.

The alginate-bio-cellulose mixture was further ground and mixed at 13,500 rpm for 3 minutes using a homogenizer. The alginate-bio-cellulose mixture was put in a frame, frozen at −80° C. for 24 hours, and then, freeze-dried for 48 hours. To cross-link the alginate, 1 part by weight of CaCl₂ relative to 100 parts by weight of the composition was treated. To remove CaCl₂, the alginate-bio-cellulose mixture was washed with diluted water for two hours, frozen at −80° C. for 24 hours, and then, freeze-dried for 48 hours to manufacture the scaffold.

To manufacture an agar-bio-cellulose composite scaffold and a carrageenan-bio-cellulose composite scaffold, 2 parts by weight of each agar and carrageenan relative to 100 parts by weight of the composition were dissolved in distilled water at 80° C. The dissolved agar and carrageenan were mixed with various concentrations of bio-cellulose powder, and the mixture was further ground and mixed at 13,500 rpm for three minutes using a homogenizer. The agar-bio-cellulose and carrageenan-bio-cellulose mixtures were put in frames and frozen at −80° C. for 24 hours, and then, freeze-dried for 48 hours to manufacture the scaffolds.

### Experimental Example 1. Culture of Muscle Stem Cells on Bio-Cellulose Mixed Scaffold

To evaluate whether the bio-cellulose powder mixed scaffold produced by the method of the embodiment 4 is more suitable for muscle cell cultivation than the scaffold manufactured solely from hydrogel materials such as alginate, agar, or carrageenan, muscle stem cells were seeded onto the bio-cellulose powder mixed scaffold. The muscle stem cells, derived from non-human animal sources such as cattle, chicken, or pig, were directly isolated through the primary cell culture.

After seeding the muscle stem cells onto each scaffold, additional culture medium was added after 3 hours of the culture to enhance adhesion. The muscle stem cells were cultured on the scaffold for a total of 96 hours. To verify cell culture on the scaffold, the survival of muscle stem cells for 24 hours and 96 hours was checked using the LIVE & DEAD assay (Thermofisher, L3223), which stains living cells green and dead cells red. After the culture, through fluorescent staining photographs for 24 hours and 96 hours, the culture capability of muscle stem cells was assessed.

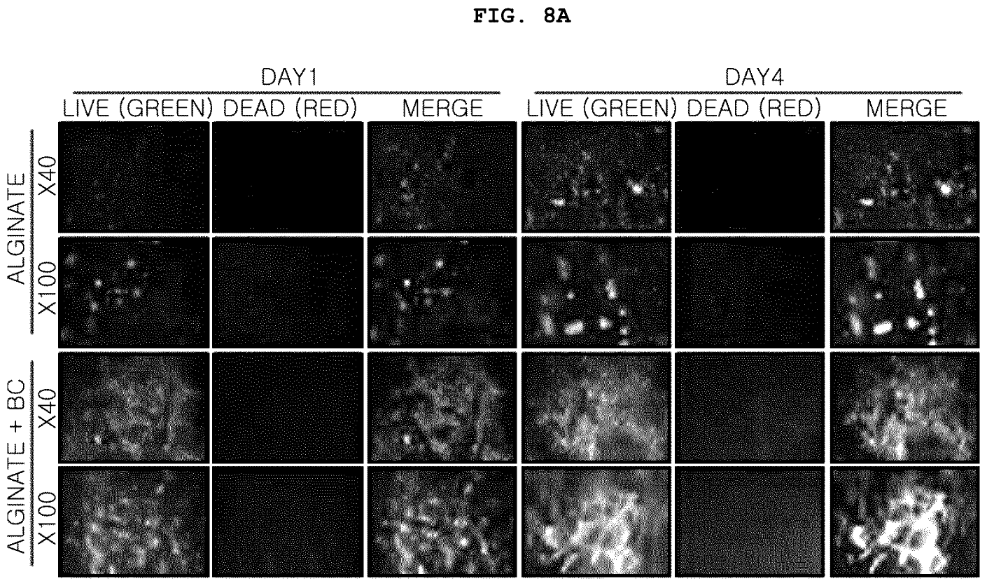

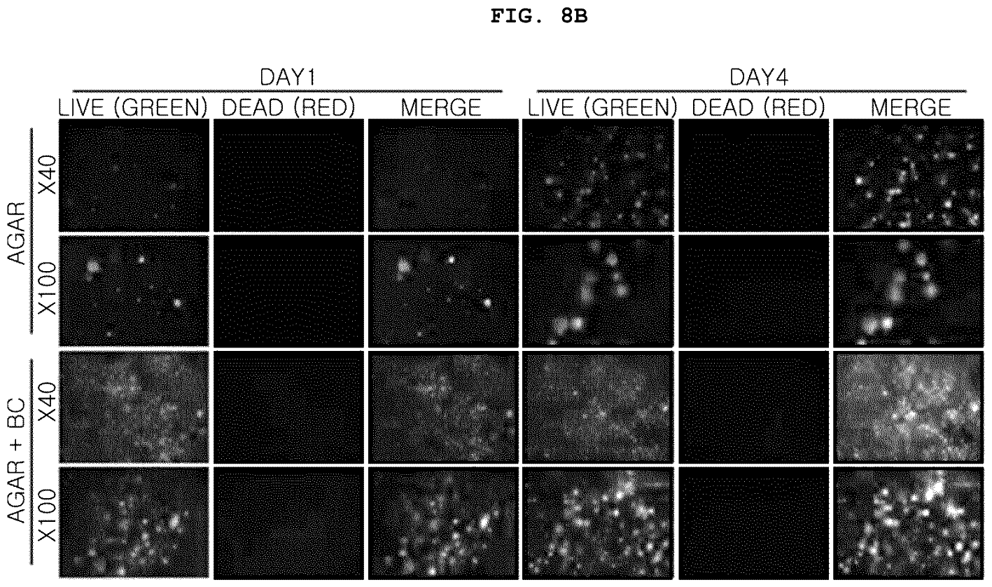

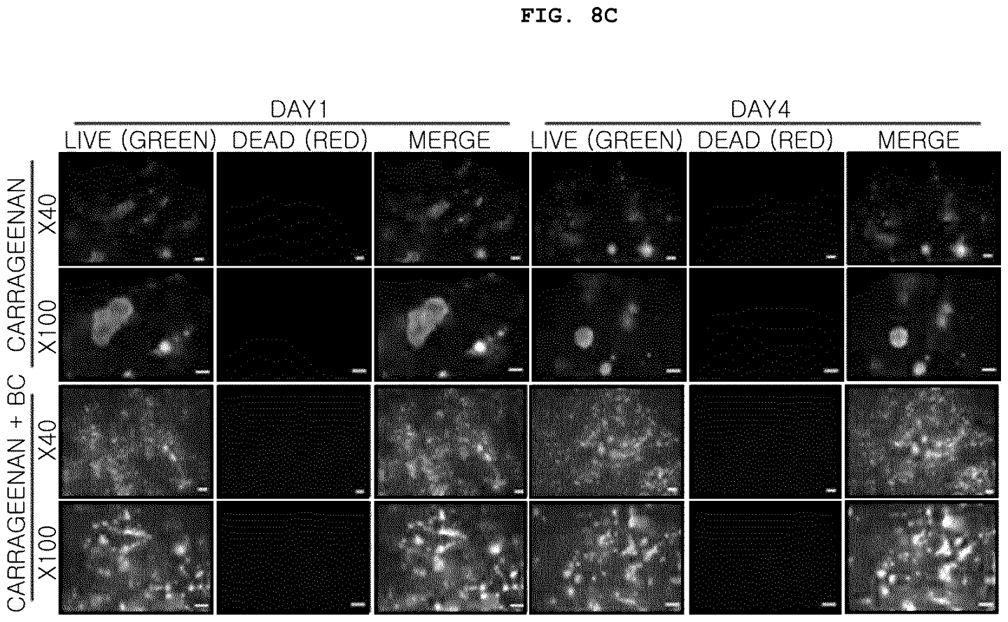

The results showed that the scaffolds manufactured from alginate, agar, or carrageenan alone did not show green-stained cells since there were no cell adhesion and cell culture, but all of the scaffolds containing bio-cellulose powder showed cell adhesion and cell culture (FIGS. 8A to 8C).

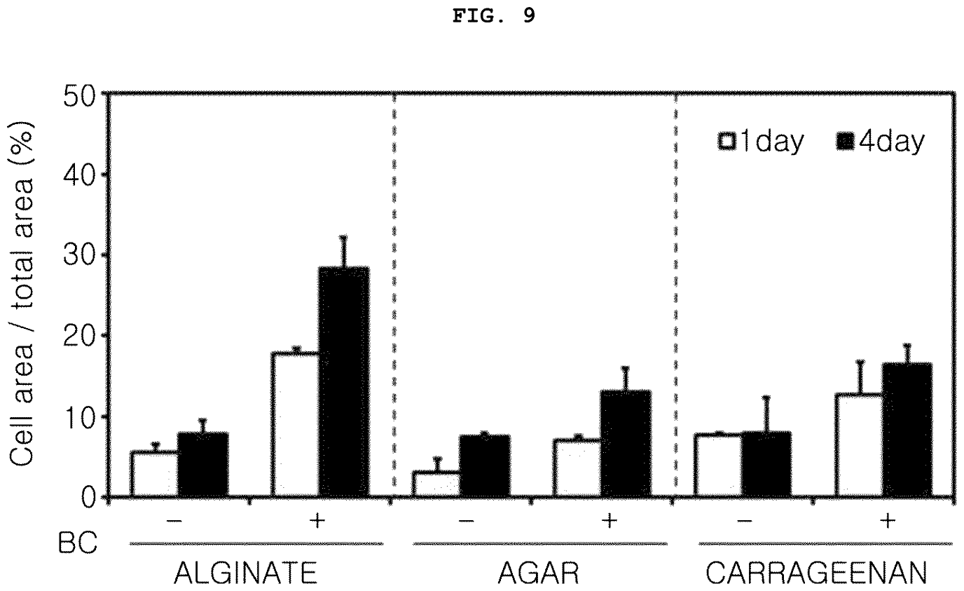

Additionally, tracking cell cultivation capability through the ratio of fluorescent staining to total area revealed that the addition of bio-cellulose powder significantly enhanced cell cultivation capability (FIG. 9).

Accordingly, it showed that the addition of bio-cellulose powder made cell adhesion and culture of the scaffolds manufactured of alginate, agar, or carrageenan possible.

### Experimental Example 2. Cross-Sectional Analysis of Bio-Cellulose Mixed Scaffold

To further verify that the culture results of muscle stem cells through the LIVE & DEAD assay was obtained through addition of bio-cellulose powder, the cross-sectional analysis of the bio-cellulose mixed scaffold was conducted.

To analyze the cross-section of the bio-cellulose mixed scaffold manufactured by the method of the embodiment 4, each of the bio-cellulose mixed scaffolds was platinum-coated for 60 seconds at 20 mA, and the cross-section of each coated scaffold was observed using scanning electron microscopy (SEM).

The cross-sections of the scaffolds containing 2 parts by weight of alginate, agar, or carrageenan relative to 100 parts by weight of the composition were regular and had predetermined pore sizes.

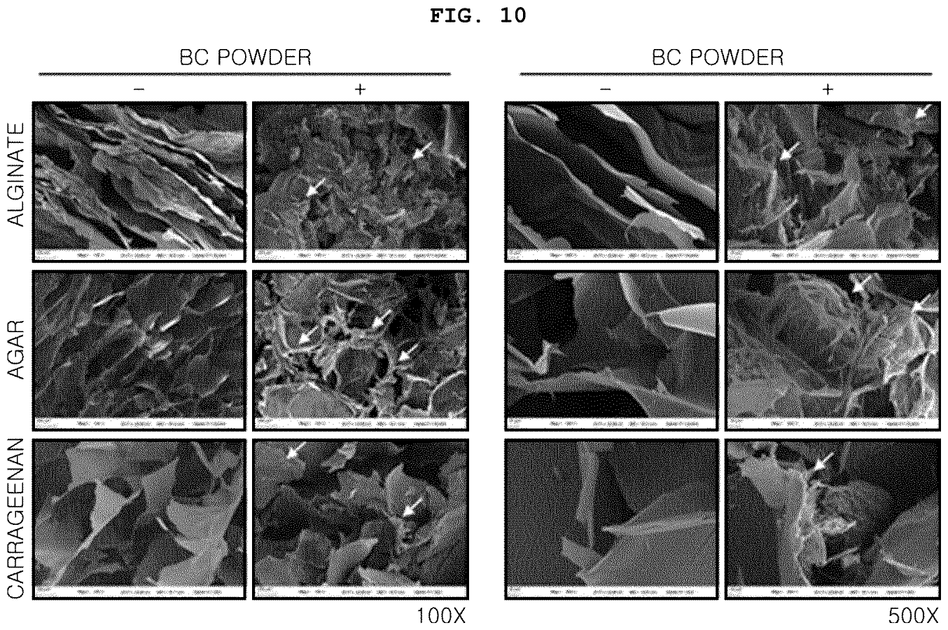

As a result of analysis of the cross-sections of the scaffolds containing 2 parts by weight of alginate, agar, or carrageenan and 2 parts by weight of bio-cellulose powder relative to 100 parts by weight of the composition, it was found that the bio-cellulose was appropriately positioned between the scaffolds manufactured of alginate, agar, or carrageenan. As a result of analysis of the cross-sections of the bio-cellulose mixed scaffolds, it was found that the bio-cellulose powder positioned between the alginate, agar, or carrageenan scaffolds made the adhesion and culture of muscle stem cells possible (FIG. 10).

### Experimental Example 3. Texture Comparison of Bio-Cellulose Mixed Scaffold According to Bio-Cellulose Powder Addition

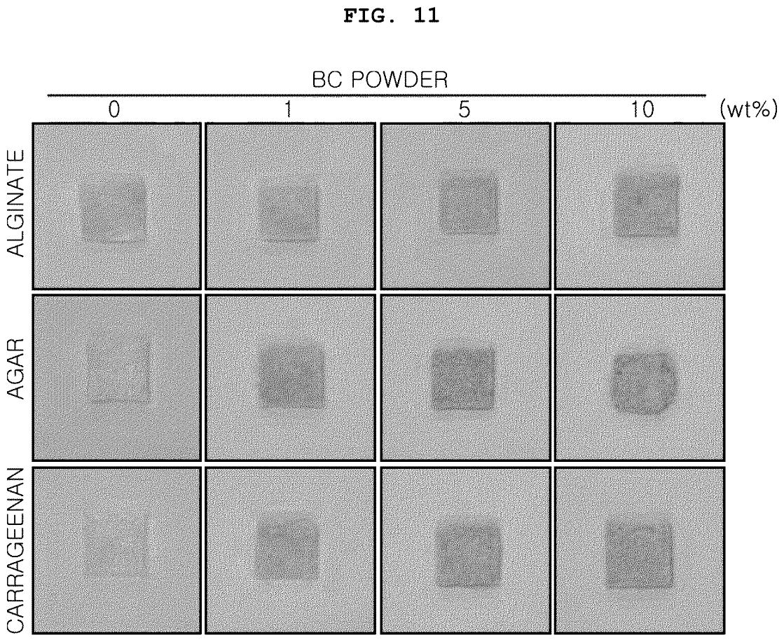

To determine whether the addition of bio-cellulose powder allows for the scaffold capable of adhesion and culture of muscle stem cells to adjust the texture according to addition amounts of bio-cellulose powder, relative to 100 parts by weight of the composition, 0, 1, 5, and 10 parts by weight of bio-cellulose powder was mixed with 2 parts by weight of alginate, agar, or carrageenan to manufacture mixed scaffolds, and then, physical property changes were observed (FIG. 11).

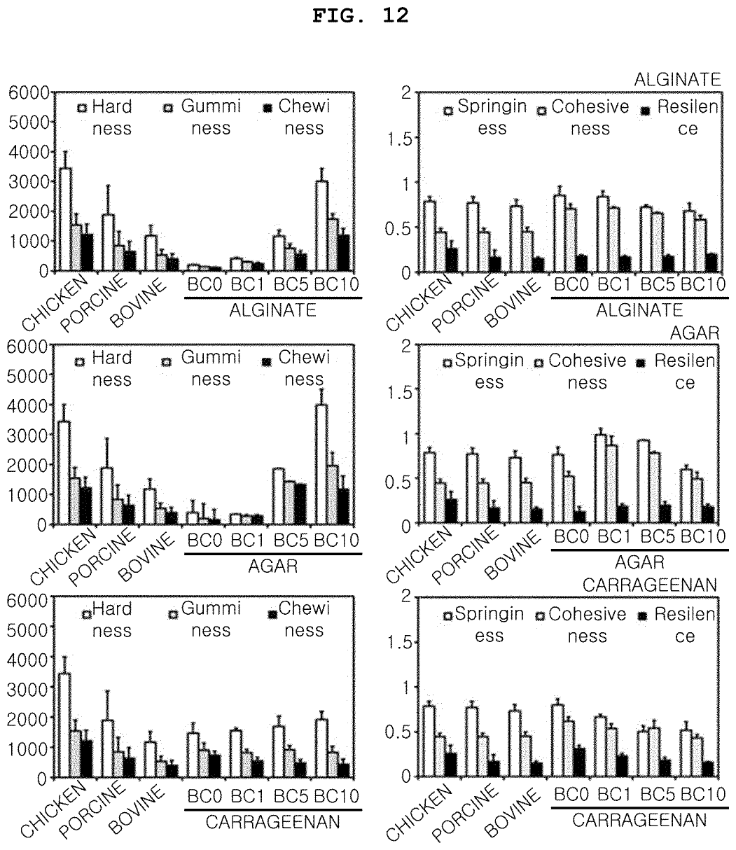

As a result, it was confirmed that, according to the concentration of bio-cellulose powder, as hardness, gumminess, and chewiness were increased, the strength of the manufactured scaffolds was increased, but springiness, cohesiveness, and resilience were decreased (FIG. 12).

Additionally, it was found that the result values for hardness, gumminess, chewiness, springiness, cohesiveness, and resilience of chicken, beef, and pork were all included within the range of result values adjusting the bio-cellulose powder content (FIG. 12).

Accordingly, the above indicates that scaffolds manufactured by mixing 1 to 10 parts by weight of bio-cellulose powder with 2 parts by weight of alginate, agar, or carrageenan, relative to 100 parts by weight of the composition, can replicate the texture of chicken, beef, and pork.

Based on the analysis of texture characteristics, the physical properties of the bio-cellulose composite support can be adjusted according to the addition concentration of bio-cellulose powder, and the addition of bio-cellulose powder can replicate the specific textures of various meats and various meat parts.

The embodiments of the invention have been described with reference to the attached drawings, but it should be understood by those skilled in the art that the present disclosure can be realized in other specific forms without departing from the spirit or essential characteristics. Therefore, the described embodiments should be considered in all respects as illustrative and not restrictive.

### ADVANTAGEOUS EFFECTS

The present disclosure provides a scaffold based on microbial-derived bio-cellulose, provides new hydrogel manufactured by mixing powdered microbial-derived bio-cellulose with traditional hydrogel materials, and can be usefully used as a scaffold for producing cultured meat due to excellent cell adhesion and physical properties.

1. A composition for manufacturing a cultured meat scaffold, which contains bio-cellulose powder derived from Gluconacetobacter sp., a natural polymer gelling agent, and a gelling accelerator, comprising:
relative to 100 parts by weight of the composition, 1 to 20 parts by weight of bio-cellulose powder, 1 to 5 parts by weight of natural polymer gelling agent, and 1 to 5 parts by weight of gelling accelerator,
wherein the natural polymer gelling agent is any one selected from a group o consisting of alginate, agar, and carrageenan.

2. The composition according to claim 1, wherein the gelling accelerator is one or more metal salts selected from a group consisting of potassium chloride, calcium chloride, magnesium chloride, and calcium lactate.

3. The composition according to claim 1, wherein the bio-cellulose powder is obtained by freezing bio-cellulose and grinding the bio-cellulose at 10,000 to 15,000 rpm.

4. The composition according to claim 3, wherein the bio-cellulose is produced in a medium containing plant peptone.

5. The composition according to claim 4, wherein the plant peptone is one or more types selected from a group consisting of soy peptone, wheat peptone, broadbean peptone, potato peptone, pea peptone, papainic soy peptone, and lupin peptone.

6. The composition according to claim 3, wherein the bio-cellulose is produced in a medium containing glucose as a carbon source.

7. A cultured meat scaffold manufactured from the composition of claim 1.

8. The cultured meat scaffold according to claim 7, wherein as the concentration of bio-cellulose powder in the composition increases, the hardness, gumminess, and chewiness of the cultured meat scaffold increase.

9. The cultured meat scaffold according to claim 7, wherein as the concentration of bio-cellulose powder in the composition increases, the elasticity, cohesiveness, and resilience of the cultured meat scaffold decrease.

10. A manufacturing method of a culture meat scaffold comprising:
(a) obtaining bio-cellulose by static culturing of Gluconacetobacter sp. in a medium containing peptone;
(b) washing the bio-cellulose obtained in operation (a) with distilled water and drying the washed bio-cellulose;
(c) freezing the dried bio-cellulose, and crushing the bio-cellulose at 10,000 to 15,000 rpm to obtain bio-cellulose powder;
(d) mixing the bio-cellulose powder with a natural polymer gelling agent; and
(e) freeze-drying the product of operation (d) and adding a gelation accelerator,
wherein relative to 100 parts by weight of the product obtained by addition of the gelation accelerator of operation (e), 1 to 20 parts by weight of bio-cellulose powder, 1 to 5 parts by weight of natural polymer gelling agent, and 1 to 5 parts by weight of gelling accelerator, and the natural polymer gelling agent is any one selected from a group consisting of alginate, agar, and carrageenan.

11. The manufacturing method according to claim 10, wherein the peptone is one or more types selected from a group consisting of soy peptone, wheat peptone, broadbean peptone, potato peptone, pea peptone, papainic soy peptone, and lupin peptone.

12. Cultured meat produced by non-human animal-derived cells on the cultured meat scaffold manufactured by the method of claim 10.

13. The cultured meat according to claim 12, wherein the non-human animal-derived cells are one or more types selected from a group consisting of muscle stem cells, muscle cells, and their progenitors.

14. The cultured meat according to claim 12, wherein the non-human animals are selected from a group consisting of cattle, sheep, pigs, poultry, crustaceans, and fish.
